SEQUENCE LISTING (9 sequences)

SEQ ID NO: 1 - DNA, 1824 bp, Homo sapiens
ggcacgaggaagaggctgactgtacgttccttctactctggcaccactctccaggctgccatggggcccagcacccctctcctcatcttgttccttttgtcatggtcgggacccctccaaggacagcagcaccaccttgtggagtacatggaacgccgactagctgctttagaggaacggctggcccagtgccaggaccagagtagtcggcatgctgctgagctgcgggacttcaagaacaagatgctgccactgctggaggtggcagagaaggagcgggaggcactcagaactgaggccgacaccatctccgggagagtggatcgtctggagcgggaggtagactatctggagacccagaacccagctctgccctgtgtagagtttgatgagaaggtgactggaggccctgggaccaaaggcaagggaagaaggaatgagaagtacgatatggtgacagactgtggctacacaatctctcaagtgagatcaatgaagattctgaagcgatttggtggcccagctggtctatggaccaaggatccactggggcaaacagagaagatctacgtgttagatgggacacagaatgacacagcctttgtcttcccaaggctgcgtgacttcacccttgccatggctgcccggaaagcttcccgagtccgggtgcccttcccctgggtaggcacagggcagctggtatatggtggctttctttattttgctcggaggcctcctggaagacctggtggaggtggtgagatggagaacactttgcagctaatcaaattccacctggcaaaccgaacagtggtggacagctcagtattcccagcagaggggctgatccccccctacggcttgacagcagacacctacatcgacctggcagctgatgaggaaggtctttgggctgtctatgccacccgggaggatgacaggcacttgtgtctggccaagttagatccacagacactggacacagagcagcagtgggacacaccatgtcccagagagaatgctgaggctgcctttgtcatctgtgggaccctctatgtcgtctataacacccgtcctgccagtcgggcccgcatccagtgctcctttgatgccagcggcaccctgacccctgaacgggcagcactcccttattttccccgcagatatggtgcccatgccagcctccgctataacccccgagaacgccagctctatgcctgggatgatggctaccagattgtctataagctggagatgaggaagaaagaggaggaggtttgaggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgtattgcaacattttgcattaaaaggaaaatccactgcaaaaaaaaaaaaaaaaaaaaaaaaaaaaaaaaaaaaaaaaaaaaaaaaaaaaaaaaaaaa

SEQ ID NO: 2 - protein, 406 aa, Homo sapiens
MGPSTPLLILFLLSWSGPLQGQQHHLVEYMERRLAALEERLAQCQDQSSRHAAELRDFKNKMLPLLEVAEKEREALRTEADTISGRVDRLEREVDYLETQNPALPCVEFDEKVTGGPGTKGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEV

SEQ ID NO: 3 - RNA, 19 nt, Rattus rattus
gauccugaagcgguuuggu

SEQ ID NO: 4 - RNA, 19 nt, Rattus rattus
ugagaaauacgauauggug

SEQ ID NO: 5 - RNA, 19 nt, Rattus rattus
gaucuacguguuagacggc

SEQ ID NO: 6 - RNA, 21 nt, Rattus rattus
agaaacuuggcuagacacaaa

SEQ ID NO: 7 - RNA, 21 nt, Rattus rattus
agauggaaaauaggagagugc

SEQ ID NO: 8 - protein, 18 aa, Rattus rattus
CQDQSSRHAAELRDFKNK

SEQ ID NO: 9 - protein, 16 aa, Rattus rattus
LDPQTLDTEQQWDTPC

(19) United States
(12) Patent Application Publication
(10) Pub. No.: US 2008/0124317 A1
(43) Pub. Date: May 29, 2008

(54) Diagnos and Treatment of Fibrosis Related Pathology

(76) Inventors: Orna Mor, Kiryat Ono (IL); Alexander Faerman, Gan Yavne (IL); Elena Feinstein, Rechovot (IL)

Correspondence Address: COOPER & DUNHAM, LLP, 1185 AVENUE OF THE AMERICAS, NEW YORK, NY, 10036, US

(21) Appl. No.: 10/562,177

(22) PCT Filed: Jun. 24, 2004
(86) PCT No.: PCT/IL04/00565

§ 371 Date: Dec. 22, 2005

Related U.S. Application Data
(60) Provisional application No. 60/482,783, filed on Jun. 25, 2003.

(51) Int. Cl. A61K 39/395; A61K 31/7105; A61P 19/10; A61P 19/02

(52) U.S. Cl. 424/130.1; 514/44

(57) ABSTRACT

This application is directed to a method for the treatment of fibrosis, preferably liver fibrosis, pulmonary fibrosis and cardiac fibrosis, in a subject in need of such treatment comprising administering to said subject an amount of an inhibitor of HNOEL-iso polypeptide sufficient to effect a substantial inhibition of the HNOEL-iso polypeptide so as to thereby treat the subject. This application is also directed to a process of obtaining a compound which inhibits human HNOEL-iso polypeptide. This application is also directed to use of a compound identified by that process in the preparation of a medicament for therapy of disease, in particular fibrosis, and to diagnostic aspects related to HNOEL-iso polypeptide.

### FIELD OF THE INVENTION

The present invention relates to the identification and isolation of polynucleotides, the expression of which is altered in various fibrosis related pathologies, and use of these isolated polynucleotides as probes for diagnosis, for screening of treatment modalities and as targets for modulation in fibrosis in general, and for liver, pulmonary and cardiac fibrosis in particular.

### BACKGROUND OF THE INVENTION

### Fibrotic Diseases

Fibrotic diseases are all characterized by the excess production of a fibrous material called the extracellular matrix, which contributes to abnormal changes in tissue architecture and interferes with normal organ function. Millions of people world-wide suffer from these chronic diseases, that are widely prevalent, debilitating and often life threatening, but no effective treatment is currently available.

The human body responds to trauma and injury by scarring. Fibrosis, a type of disorder characterized by excessive scarring, occurs when the normal wound healing response is disturbed. During fibrosis, the wound healing response continues causing an excessive production and deposition of collagen.

Although fibrotic disorders can be acute or chronic, the disorders share a common characteristic of excessive collagen accumulation and an associated loss of function when normal tissue is replaced with scar tissue.

Fibrosis results from diverse causes, and may be established in various organs. Cirrhosis, pulmonary fibrosis, sarcoidosis, keloids, hypertension and renal fibrosis, are all chronic diseases that induce a progressive fibrosis thereby causing a continuous loss of tissue function.

Acute fibrosis (usually with a sudden and severe onset and of short duration) occurs as a common response to various forms of trauma including accidental injuries (particularly injuries to the spine and central nervous system), infections, surgery (cardiac scarring following heart attack), burns, environmental pollutants, alcohol and other types of toxins, acute respiratory distress syndrome, radiation and chemotherapy treatments. All tissues damaged by trauma are prone to scar and become fibrotic, particularly if the damage is repeated. Deep organ fibrosis is often extremely serious because the progressive loss of organ function leads to morbidity, hospitalization, dialysis, disability and even death. Fibrotic diseases or diseases in which fibrosis is evident (fibrotic related pathologies) include pulmonary fibrosis, interstitial lung disease, human fibrotic lung disease, liver fibrosis, cardiac fibrosis, macular degeneration, retinal and vitreal retinopathy, myocardial fibrosis, Grave's opthalmopathy, drug induced ergotism, cardiovascular disease, atherosclerosis/restenosis, keloids and hypertrophic scars, cancer, Alzheimer's disease, scarring, scleroderma, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease, including collagenous colitis.

For further information on different types of fibrosis see: Molina V, Blank M, Shoenfeld Y. (2002), “Fibrotic diseases”, Harefuah, 141(11): 973-8, 1009; Yu L, Noble N A, Border Wash. (2002), “Therapeutic strategies to halt renal fibrosis”, Curr Opin Pharmacol. 2(2): 177-81; Keane W F, Lyle P A. (2003), “Recent advances in management of type 2 diabetes and nephropathy: lessons from the RENAAL study”, Am J Kidney Dis. 41(3 Suppl 2): S22-5; Bohle A, Kressel G, Muller Calif., Muller Ga. (1989), “The pathogenesis of chronic renal failure”, Pathol Res Pract. 185(4):421-40; Kikkawa R, Togawa M, Isono M, Isshiki K, Haneda M. (1997), “Mechanism of the progression of diabetic nephropathy to renal failure”, Kidney Int Suppl. 62:S39-40; Bataller R, Brenner D A. (2001), “Hepatic stellate cells as a target for the treatment of liver fibrosis”, Semin Liver Dis. 21 (3):437-51; Gross T J, Hunninghake G W, (2001) “Idiopathic pulmonary fibrosis”, N Engl J Med. 345(7):517-25; Frohlich E D. (2001) “Fibrosis and ischemia: the real risks in hypertensive heart disease”, Am J Hypertens; 14(6 Pt 2):194S-199S.

### Liver Fibrosis

Liver fibrosis (LF) is a generally irreversible consequence of hepatic damage of several etiologies. In the Western world, the main etiologic categories are: alcoholic liver disease (30-50%), viral hepatitis (30%), biliary disease (5-10%), primary hemochromatosis (5%), and drug-related and cryptogenic cirrhosis (unknown etiology 10-15%). There are also cases of Wilson's disease, α₁-antitrypsin deficiency and other rare diseases.

The end stage of chronic liver disease is characterized by formation of fibrous septa (scars) replacing multiple adjacent lobules, followed by parenchymal nodules created by encircled hepatocytes and eventually disruption of the architecture of the entire liver. Liver cirrhosis, the end stage of liver fibrosis, frequently requires liver transplantation and is among the top ten causes of death in the Western world.

Hepatic stellate cells (HSC) are one of the key cell types involved in the initiation and progression of liver fibrosis. In response to cytokines released by damaged hepatocytes, HSC proliferate and undergo activation and transformation from vitamin A-storing cells into collagen-producing myofibroblasts.

Anti-inflammatory agents, inhibition of activation of stellate cells, stimulation of growth of hepatocytes and inhibition of post translational modification of collagen have all been used to treat liver fibrosis. However, due to the lack of selective targeting, these treatments suffer from many drawbacks including severe adverse side-effects. For more information on liver fibrosis see: Friedman S L. (2003), “Liver fibrosis—from bench to bedside”, J. Hepatol. 38 Suppl 1:S38-53; Albanis E, Safadi R, Friedman S L. (2003), “Treatment of hepatic fibrosis: almost there”, Curr Gastroenterol Rep. 5(1):48-56.

### Pulmonary Fibrosis

Interstitial pulmonary fibrosis (IPF) is scarring of the lung caused by a variety of inhaled agents including mineral particles, organic dusts, and oxidant gases. The disease afflicts millions of individuals worldwide, and there are no effective therapeutic approaches. A major reason for the lack of useful treatments is that few of the molecular mechanisms of disease have been defined sufficiently to design appropriate targets for therapy (Lasky J A., Brody A R. (2000), “Interstitial fibrosis and growth factors”, Environ Health Perspect.; 108 Suppl 4:751-62).

The pathogenesis of pulmonary fibrosis includes endothelial and epithelial cell injury, production of inflammatory cells and their mediators, and fibroblast activation, and is believed to be related to a dysregulation in cross-talk between inflammatory and structural cells, mediated by various cytokines, chemokines and growth factors, which are responsible for the maintenance of tissue homeostasis and which coordinate the response to injury (Kelly M, Kolb M, Bonniaud P, Gauldie J. (2003), “Re-evaluation of fibrogenic cytokines in lung fibrosis”, Curr Pharm Des. 9(1):39-49).

Conventional therapy consisting of glucocorticoids or cytotoxic drugs is usually ineffective in preventing progression of the disease. It is believed that further understanding of the molecular mechanisms of endothelial and epithelial cell injury, inflammatory reaction, fibroblast proliferation, collagen deposition and lung repair, is necessary for the development of effective treatments against pulmonary fibrosis (Kuwano K, Hagimoto. N, Hara N. (2001), “Molecular mechanisms of pulmonary fibrosis and current treatment”, Curr Mol. Med. 1(5):551-73).

### Cardiac Fibrosis

Heart failure is unique among the major cardiovascular disorders in that it alone is increasing in prevalence while there has been a striking decrease in other conditions. Some of this can be attributed to the aging of the populations of the United States and Europe. The ability to salvage patients with myocardial damage is also a major factor, as these patients may develop progression of left ventricular dysfunction due to deleterious remodelling of the heart.

The normal myocardium is composed of a variety of cells, cardiac myocytes and noncardiomyocytes, which include endothelial and vascular smooth muscle cells and fibroblasts. (Weber K T. (2000), “Fibrosis and hypertensive heart disease”, Curr Opin Cardiol. 15(4):264-72).

Structural remodeling of the ventricular wall is a key determinant of clinical outcome in heart disease. Such remodeling involves the production and destruction of extracellular matrix proteins, cell proliferation and migration, and apoptotic and necrotic cell death. Cardiac fibroblasts are crucially involved in these processes, producing growth factors and cytokines that act as autocrine and paracrine factors, as well as extracellular matrix proteins and proteinases. Recent studies have shown that the interactions between cardiac fibroblasts and cardiomyocytes are essential for the progression of cardiac remodeling of which the net effect is deterioration in cardiac function and the onset of heart failure (Manabe I, Shindo T, Nagai R. (2002), “Gene expression in fibroblasts and fibrosis: involvement in cardiac hypertrophy”, Circ Res. 13; 91(12):1103-13).

The use of agents to block the renin-angiotensin-aldosterone and sympathetic nervous systems has been shown to inhibit (and sometimes even reverse) cardiac remodelling and to improve the clinical course of patients with cardiac dysfunction. However, drugs aiming at direct inhibition or reduction of fibrosis are not yet available (Greenberg B. (2001), “Treatment of heart failure: state of the art and prospectives”, J Cardiovasc Pharmacol. 38 Suppl 2:S59-63).

### Nephropathy

The term “nephropathy” encompasses all clinical-pathological changes in the kidney which may result in kidney fibrosis and/or glomerulosclerosis and/or chronic renal insufficiency, and can cause end stage renal disease. Note that the terms “chronic renal insufficiency” (CRF) and “chronic renal failure” (CRF) are used interchangeably throughout this application. Diabetic nephropathy, hallmarks of which are glomerulosclerosis and renal fibrosis, is the single most prevalent cause of end-stage renal disease in the modern world, and diabetic patients constitute the largest population on dialysis. Such therapy is costly and far from optimal. Transplantation offers a better outcome but suffers from a severe shortage of donors. More targeted therapies against diabetic nephropathy (as well as against other types of kidney pathologies) are not developed, since molecular mechanisms underlying these pathologies are largely unknown. Identification of an essential functional target gene that is modulated in the disease and affects the severity of the outcome of diabetes nephropathy has a high diagnostic as well as therapeutic value.

It is known in the art that many pathological processes in the kidney eventually culminate in similar or identical morphological changes, namely glomerulosclerosis and fibrosis. Human kidney disease may evolve from various origins including glomerular nephritis, nephritis associated with systemic lupus, cancer, physical obstructions, toxins, metabolic disease and immunological diseases, all of which culminate in renal fibrosis. The meaning of this phenomenon is that different types of insults converge on the same single genetic program resulting in two hallmarks of fibrosis: the proliferation of fibroblasts and overproduction by them of various protein components of connective tissue. In addition, thickening of the basal membrane in the glomeruli accompanies interstitial fibrosis and culminates in glomerulosclerosis. Genes encoding proteins that are involved in kidney fibrosis and glomerulosclerosis may be roughly divided into two groups:
1. Genes, the expression of which leads to the triggering of proliferation of fibroblasts and overproduction by them of various protein components of connective tissue. These may be specific to different pathological conditions;
2. Genes, the expression of which leads to the execution of the “fibrotic or sclerotic programs”. These may be common to all renal pathologies leading to fibrosis and glomerulosclerosis.

The identification of genes that belong to the second group should contribute to the understanding of molecular mechanisms that accompany fibroblast and mesangial cell proliferation and hypersecretion, and may constitute genetic targets for drug development, aimed at preventing renal failure. Application of such drugs is expected to suppress, retard, prevent, inhibit or attenuate progression of fibrosis and glomerulosclerosis.

A useful way to assess the development of renal diseases involving fibrosis and glomerulosclerosis is to characterize gene expression in established animal models of kidney diseases. Examples of such models include without limitation: (i) fa/fa rats—animals genetically deficient in leptin receptor that develop insulin resistant diabetes (type II diabetes) with progressive diabetic nephropathy, and (ii) GK rats—which are genetically manipulated, NIDDM phenotype rats. Another animal model in which mainly kidney fibrosis is evident, but without a background of diabetes, is unilateral ureteral obstruction (UUO) in which interstitial fibrosis is rapid and occurs within days following the obstruction. 5/6 nephrectomy is another useful animal model for chronic renal insufficiency (CR1) in which fibrosis is evident.

Additional aspects of research may be based on an in vitro model system involving culture of human fibroblasts in vitro under conditions mimicking various parameters of the cell microenvironment existing in CRI and fibrosis. These conditions include treatment with high concentrations of glucose (modeling hyperglycemia), low concentrations of glucose, hypoxia (both modeling ischemic conditions that develop in the kidney following fibrosis and glomerulosclerosis), and TGF-β-one of the recognized pathogenic factors in fibrosis. Such in vitro model systems may complement the animal models in several important aspects: First, the system is fibroblast-specific; accordingly, none of the interferences often found in complex tissues that contain many cell types are present. Second, the cells are of human origin, unlike the animal models. Furthermore, the insults are specific and of various concentrations and duration, thus enabling the investigation of both acute and chronic responses

### HNOEL-iso

HNOEL-iso a 1.8 kb mRNA encoding ER localized protein of 406 amino acids that contains the olfactomedin (OLF) domain (starts at amino acid at position 135 and ends at position 401). The function of this protein is unknown, however, it is a part of a protein family having olfactomedin domains. These olfactomedin-related proteins are secreted glycoproteins with conserved C-terminal motifs. All these proteins show highly specific expression patterns. One of these proteins, BMZ, was found to be expressed in the Golgi apparatus of glomerular podocytes. Other olfactomedin-related proteins are expressed in mucous tissues. Therefore, it has been suggested that these proteins might play a role in regulating physical properties of the extracellular environment. Another family member, the TIGR/myocilin protein has been demonstrated to be involved in ocular hypertension. Mutations in the TIGR gene are concomitant with steroid-induced ocular hypertension.

The HNOEL-iso polypeptide is secreted from the cells. Additionally, it has been found to be expressed in breast and melanoma.

### Structural Information

### Nucleotide Sequence (SEQ ID. NO:1):

gi|14602834|gb|BC009920.1|BC009920 Homo sapiens, HNOEL-iso protein, clone MGC:2896 IMAGE:3010136, mRNA, complete cds;
Also published as: gi|9910269|ref|NM_{—}020190.1| Homo sapiens HNOEL-iso protein (HNOEL-iso), mRNA).
   Protein sequence (SEQ ID NO:2):
gi|14602835|gb|AAH09920.1|AAH09920 HNOEL-iso protein [Homo sapiens]

The disclosures of the following patents and patent applications in their entireties are hereby incorporated by reference into this application in order to more fully describe the state of the art to which this invention pertains: WO 00/12708, WO 00/32221, WO 00/55375, WO 0078808, WO 00/78961, WO 01/54477, EP 1 067 182.

### SUMMARY OF THE INVENTION

The main object of the present invention is the identification and isolation of novel genetic targets that may be used for development of drugs to treat fibrosis and fibrosis related pathologies in general, and for pulmonary fibrosis, interstitial lung disease, human fibrotic lung disease, liver fibrosis, renal fibrosis, cardiac fibrosis, macular degeneration, retinal and vitreal retinopathy, myocardial fibrosis, Grave's opthalmopathy, drug induced ergotism, cardiovascular disease, atherosclerosis, Restenosis, Keloids and Hypertrophic Scars, cancer, Alzheimer's disease, scarring, scleroderma, Glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease including collagenous colitis, in particular, and usage of such targets as a tool for diagnostic and prognostic applications.

The present invention provides novel targets for development of novel therapeutic and diagnostic means, via large-scale microarray-based analysis of gene expression in fibrotic models in vivo and in vitro. In one embodiment, the present invention identifies up- or down-regulator (responder) genes for gene therapy, diagnostics and therapeutics that have direct causal relationships between fibrotic diseases and their related pathologies. More preferably, the present invention identifies the HNOEL-iso gene as a modulator gene in fibrosis and in fibrosis related diseases.

The present invention further provides a process referred to herein as a screening assay for identifying modulators, i.e., candidate or compounds or agents including but not limiting to neutralizing antibodies, peptides, peptido-mimetics, small molecules and other drugs, which bind to HNOEL-iso or have an effect on HNOEL-iso expression or on HNOEL-iso activity.

The compound or agent discovered by the above-mentioned screening assay that may affect signalling via the HNOEL-iso polypeptide can be used in various fibrosis related pathologies to modulate collagen uptake, fibronectin and/or MMP uptake, fibroblast adhesion and migration on fibrillar collagen matrices and mesangial cell proliferation and cell apoptosis. It can further be used to reduce the proliferation of fibroblasts, to inhibit the accumulation of extracellular matrix and to reduce or limit the formation of fibrotic regions in a target tissue/organ.

### BRIEF DESCRIPTION OF THE FIGURES

FIG. 1. This figure represents the nucleotide sequence of the human HNOEL-iso gene—SEQ ID NO:1.

FIG. 2. This figure represents the amino acid sequence of the human HNOEL-iso gene—SEQ ID NO:2.

FIG. 3. This figure represents a construct used to establish transgenic mice that express the HNOEL-iso gene in their kidneys. As shown in the figure, a rat HNOEL gene (r89B7F) was inserted into the Not1/Mlu1 sites of the KSPMCS vector, containing the KSP-cadherin gene promoter which is known to be tubular specific promoter. The Asc-1 fragment (5500 bp) containing the KSP promoter, the gene and the SV40 region was cut from the plasmid and injected into FVBN mouse eggs.

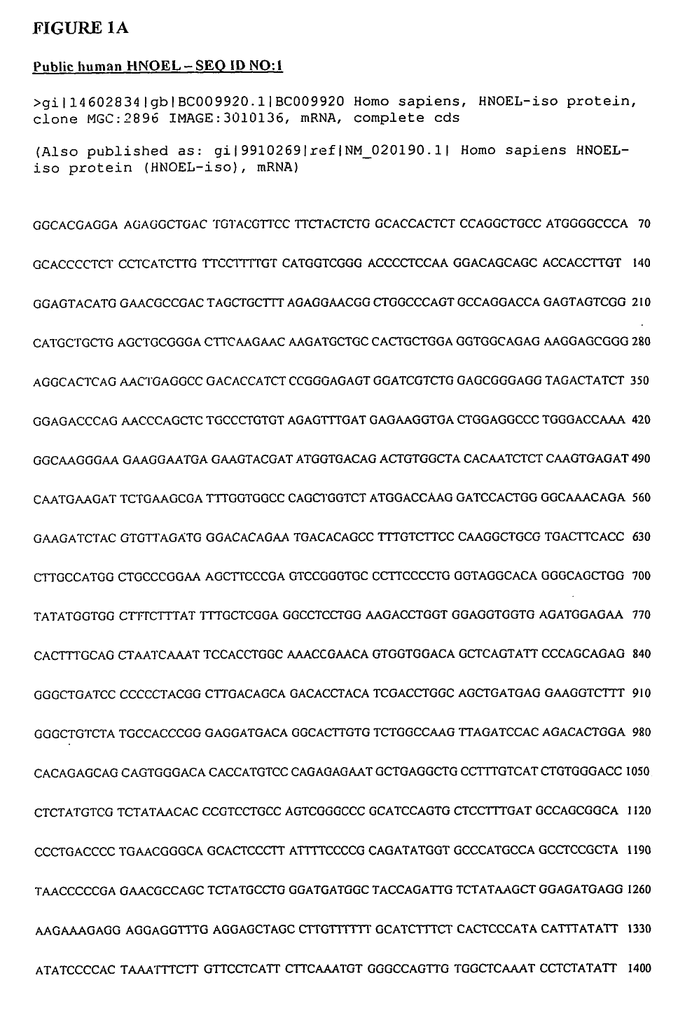

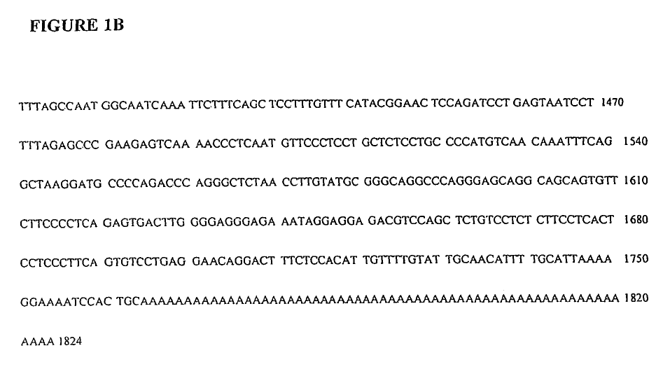

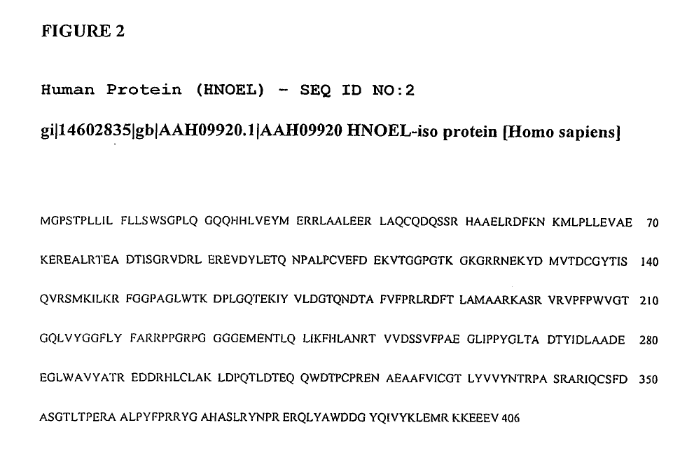

### DETAILED DESCRIPTION OF THE INVENTION

In accordance with the present invention, purified, isolated and cloned nucleic acid sequences, specifically the nucleic acid sequence that encodes the HNOEL-iso polypeptide, and having sequences as specified herein or having complementary or allelic sequence variations thereto, are disclosed as being associated with fibrosis, and more specifically with fibrosis related pathologies which include pulmonary fibrosis, interstitial lung disease, human fibrotic lung disease, liver fibrosis, cardiac fibrosis, macular degeneration, retinal and vitreal retinopathy, myocardial fibrosis, Grave's opthalmopathy, drug induced ergotism, cardiovascular disease, atherosclerosis, restenosis, keloids and hypertrophic scars, cancer, Alzheimer's disease, scarring, scleroderma, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease including collagenous colitis. In particular, the nucleic acid sequence that encodes the HNOEL-iso polypeptide has a sequence of SEQ ID NO:1 which encodes SEQ ID NO:2 herein, and in particular HNOEL is deemed to be associated with liver fibrosis, pulmonary fibrosis and cardiac fibrosis.

As used herein, the term “HNOEL-iso gene” or “HNOEL gene” is defined as any homolog of the INOEL-iso gene having preferably 90% homology, more preferably 95% homology, and even more preferably 98% homology to the amino acid encoding region of SEQ ID NO: 1 or nucleic acid sequences which bind to the HNOEL-iso gene under conditions of highly stringent hybridization, which are well-known in the art, for example, see Ausubel et al., Current Protocols in Molecular Biology, John Wiley and Sons, Baltimore, Md. (1988), updated in 1995 and 1998.

As used herein, the term “HNOEL-iso”, “HNOEL” or “HNOEL-iso polypeptide” is defined as any homolog of the HNOEL-ISO polypeptide having preferably 90% homology, more preferably 95% homology, and even more preferably 98% homology to SEQ ID NO:2, as either full-length or a fragments or a domain thereof, as a mutant or the polypeptide encoded by a spliced variant nucleic acid sequence, as a chimera with other polypeptides, provided that any of the above has the same or substantially the same biological function as the HNOEL-iso polypeptide. HNOEL-iso polypeptide, or an HNOEL-iso polypeptide homolog, may be present in different forms, including but not limited to soluble protein, membrane-bound (either in purified membrane preparations or on a cell surface), bead-bound, or any other form presenting HNOEL-iso protein or fragments and polypeptides derived thereof.

Where the sequences are partial sequences, they may be used as markers/probes for genes that are modulated in fibrosis. In general these partial sequences which are designated “Expressed Sequence Tags” (ESTs), are markers for the genes actually expressed in vivo, and are ascertained as described herein in the Examples section. Generally, ESTs comprise DNA sequences corresponding to a portion of nuclear encoded mRNA. The EST has a length that allows for polymerase chain reaction (PCR), and is used as a hybridization probe, with a unique designation for the gene with which it hybridizes (generally under conditions sufficiently stringent to require at least 95% base pairing). For a detailed description and review of ESTs and their functional utility see WO 93/00353 which is incorporated herein in its entirety by reference. WO 93/00353 further describes how the EST sequences can be used to identify the transcribed genes.

As used herein, an “interactor” is a molecule with which HNOEL-iso or an HNOEL-iso gene family member binds or interacts or activates in nature; for example, a molecule on the surface of a cell that expresses HNOEL-iso polypeptide, a molecule on the surface of a second cell or a cytoplasmic molecule. An interactor may be a ligand that is activated by HNOEL-iso alone or by HNOEL-iso as part of a complex with other components. An interactor may be a component of a signal transduction pathway that facilitates transduction of an extracellular signal from HNOEL-iso through the cell membrane and into the cell. An interactor, for example, can be a second intercellular protein that mediates downstream signaling from HNOEL-iso.

As used herein, the term “compound” or “inhibitor” is defined as comprising any small chemical molecule, antibodies, neutralizing antibodies, antisense DNA or RNA molecules, siRNA, proteins, polypeptides and peptides including peptido-mimetics and dominant negatives, and expression vectors.

In one embodiment, the invention provides assays for screening candidates or compounds or inhibitors that bind to, inhibit the activity of, or inhibit the expression level of HNOEL-iso. The compounds of the present invention can be obtained by using any of the numerous approaches in combinatorial and non-combinatorial library methods known in the art, including biological libraries (proteins, peptides, etc.), spatially addressable parallel solid phase or solution phase libraries, synthetic library methods, and natural product libraries.

The compound that inhibits the HNOEL-iso polypeptide may inhibit the expression of the polypeptide or its transcription or translation, or the polypeptide activity. This inhibitor may be inter alia a small chemical molecule which generally has a molecular weight of less than 2000 daltons, more preferably less than 1000 daltons, even more preferably less than 500 daltons. Other inhibitors may be antibodies preferably neutralizing antibodies or fragments thereof including single chain antibodies, antisense polynucleotides, antisense DNA or RNA molecules, siRNA, proteins, polypeptides and peptides including peptido-mimetics and dominant negatives, and expression vectors. These inhibitors may act as follows: small molecules may affect expression and/or activity; antibodies—may affect activity; all kinds of antisense and siRNA—may effect HNOEL-iso expression; dominant negative polypeptides and peptidomimetics—may affect activity; expression vectors may be used inter alia for delivery of antisense or dominant-negative polypeptides. Approaches have recently been developed that utilize small molecules, which can bind directly to proteins and can be used to alter protein function; for review see B. R. Stockwell, (2000) Nature Reviews/Genetics, 1, 116-125. As mentioned above, low molecular weight organic compounds can permeate the plasma membrane of target cells relatively easily and, therefore, methods have been developed for their synthesis. These syntheses, in turn, have yielded libraries that contain ligands for many proteins. Recent developments have brought a greatly increased variety of creatively selected, novel, small organic molecules that will function as powerful tools for perturbing biological systems. Such small molecules can be used to activate or inactivate specific members of a protein family.

Examples of methods for the synthesis of molecular libraries can be found in the art, for example, in DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carrell et al. (1994) Angew. Chem. Int. Ed. Engl. 33:2061; and in Gallop et al. (1994) J. Med. Chem. 37:1233.

In another aspect of the invention, HNOEL-iso polypeptide can be used as “bait protein” in a two-hybrid assay or three-hybrid assay (e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins which bind to or interact with HNOEL-iso (“HNOEL-iso-binding proteins”) and modulate HNOEL-iso activity. Such HNOEL-iso-binding proteins are also likely to be involved in the propagation of signals by HNOEL-iso as, for example, upstream or downstream elements of the HNOEL-iso signaling pathway.

The term “treatment” as used herein refers to administration of a therapeutic substance effective to ameliorate symptoms associated with a disease, to lessen the severity or cure the disease, or to prevent the disease from occurring.

### Screening Systems

The HNOEL-iso gene or polypeptide may be used in a screening assay for identifying and isolating compounds which inhibit or retard fibrosis and related pathologies, in particular pulmonary fibrosis, liver fibrosis and cardiac fibrosis.

As is well known in the art, the screening assays can be cell-based or non-cell-based. A cell-based assay is performed using eukaryotic cells such as HeLa cells. One way of running such a cell-based assay uses tetracycline-inducible (Tet-inducible) gene expression. which is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285, 1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J.Neuroscience 18, 5285 and Tang et al (1997) J. Clin. Invest 100, 1180 for measurement of kinase activity. It is possible that HNOEL-iso interacts with an enzyme and regulate its enzymatic activity through protein-protein interaction. One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

The present invention provides for a process of obtaining a compound which inhibits human HNOEL-iso polypeptide that comprises the steps of:
(i) contacting cells expressing the HNOEL-iso polypeptide with the compound;
(ii) measuring the effect of the compound on a parameter related to fibrosis; and
(iii) comparing the effect measured in step (ii) with the effect measured in the absence of the compound, a decrease in the effect measured indicating that the compound inhibits the human HNOEL-iso polypeptide.

In one embodiment, the HNOEL-iso polypeptide used in such process, comprises consecutive amino acids the sequence of which is set forth in SEQ ID NO: 2.

In another embodiment the parameter measured in such process may be the content of collagen, fibronectin, or hydroxy proline content in the cells, the proliferation rate of the cells or any extracellular matrix components in the cells.

In yet another embodiment, the cells used in such process comprise a tissue, and the parameter measured is, for example, interstitial tissue volume, total tissue volume, the degree of inflammation in the tissue or the degree of apoptosis in the tissue.

In a further embodiment of the invention, the cells that are used for said process are fibroblast cells that express the HNOEL-iso polypeptide. The fibroblast cells may be selected from liver, pulmonary and cardiac fibroblast cells.

In a different embodiment, the cells used in such process express the HNOEL-iso polypeptide as a result of having been transfected with the HNOEL-iso gene, either transiently or stably transfected.

The present invention further provides a process of obtaining a compound which inhibits a human HNOEL-iso polypeptide that comprises the steps of:
i. contacting the HNOEL-iso polypeptide with an interactor with which the HNOEL-iso polypeptide interacts specifically in vivo;
ii. contacting the HNOEL-iso polypeptide or the interactor with said compound; and
iii. measuring the effect of the compound on the interaction between HNOEL-iso polypeptide and the interactor by measuring a parameter related to fibrosis; and
iv. comparing the effect measured in step (iii) with the effect measured in the absence of the compound, a decrease in the effect measured indicating that the compound inhibits human HNOEL-iso polypeptide.

In one embodiment of the invention the HNOEL-iso polypeptide in such process comprises consecutive amino acids, the sequence of which is set forth in SEQ ID NO:2. In another embodiment, the compound obtained by such process is used in a preparation of a medicament for the therapy of fibrosis related pathologies. In yet another embodiment, the compound obtained by the process is being used in the preparation of a medicament for treatment of fibrosis in general and for liver fibrosis, pulmonary fibrosis and cardiac fibrosis in particular.

In one embodiment of the invention either the HNOEL-iso polypeptide or the interactor may be immobilized.

It is an object of the present invention to provide a process of obtaining a compound which affects the activity of a human HNOEL-iso polypeptide by screening a plurality of compounds that comprises the steps of:
(i) contacting cells expressing the HNOEL-iso polypeptide with a plurality of compounds;
(ii) measuring the effect of the compounds on a parameter related to fibrosis;
(iii) comparing the effect measured in step (ii) with the effect measured in the absence of the compounds, a decrease in the effect measured indicating that the compounds inhibit the human HNOEL-iso polypeptide; and
(iv) separately determining which compound or compounds present in the plurality inhibits the human HNOEL-iso polypeptide.

It is within the scope of the present invention to use a compound obtained according to the processes described herein, in the preparation of a medicament for treatment of fibrosis and fibrosis related pathologies as described above. The compound is preferably for the preparation of a medicament for treatment of liver fibrosis, pulmonary fibrosis and cardiac fibrosis. These drugs may also be used for treatment of osteoarthritis and treatment of osteoporosis as well as other bone diseases and also for the treatment of cardiovascular diseases.

Any of the screening assays according to the present invention can include a step of obtaining the compound (as described above) which tests positive in the assay, and can also include the further step of producing said compound as a medicament. It can also include steps of improving the compound to increase its desired activity before incorporating the improved compound into a medicament. It is considered that medicaments comprising such compounds are part of the present invention.

The present invention also provides for a process of preparing a pharmaceutical composition which comprises:
i. obtaining a compound that inhibits a human HNOEL-iso polypeptide using any of the process described herein; and
ii. admixing said compound with a pharmaceutically acceptable carrier.

In a preferred embodiment, the compound used in the preparation of a pharmaceutical composition is admixed with a carrier in a pharmaceutically effective amount.

In one embodiment of the present invention, the compound obtained in the described processes is an antibody, or siRNA or antisense RNA or a small molecule. In another embodiment, the present invention is directed to a use of these compounds in preparation of a medicament useful for the treatment of fibrosis and fibrosis related pathologies as described above and in particular for liver fibrosis, pulmonary fibrosis and cardiac fibrosis.

A pharmaceutical composition for the treatment of fibrosis comprising as an active ingredient an antibody which binds specifically to HNOEL-iso polypeptide together with a pharmaceutically acceptable carrier, is also provided.

Additionally, the present invention provides a method of regulating fibrosis-associated pathologies in a patient in need of such treatment by administering to a patient a therapeutically effective amount of at least one antisense (AS) polynucleotide or siRNA against the HNOEL nucleotide sequence or a dominant negative peptide directed against the HNOEL-iso sequences or HNOEL-iso proteins.

As used herein, “negative dominant peptide” refers to a partial cDNA sequence that encodes a part of a protein, i.e., a peptide (Herskowitz I. (1987) Nature (Review) 329(6136): 219-222). This peptide can have a function different from that of the protein from which it was derived. It can interact with a wild type protein target and inhibit its activity or it can interact with other proteins and inhibit their activity in response to the wild type target protein. Specifically, negative dominant refers to the ability of a peptide to inhibit the activity of a natural protein normally found in the cell in order to modulate the cellular phenotype, i.e., making the cell more resistant or sensitive to killing. For therapeutic intervention either the peptide itself is delivered as the active ingredient of a pharmaceutical composition or the cDNA can be delivered to the cell utilizing the same methods as for AS delivery.

The modulator is dosed and delivered in a pharmaceutically acceptable carrier as described herein below. The modulator may be an antagonist agent or regulating active ingredient. As used herein, the term “antagonist or antagonizing” is understood in its broadest sense. Antagonism can include any mechanism or treatment that results in inhibition, inactivation, blocking or reduction in gene activity or gene product. It should be noted that the inhibition of a gene or gene product may provide for an increase in a corresponding function that the gene or gene product was regulating. The antagonizing step can include blocking cellular receptors for the gene products and can include AS or siRNA treatment as discussed below.

The compounds (modulators) or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art. The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24- to 48 hrs or by continuous infusion during a period of 1-2 weeks or longer.

Antisense technology. Many reviews have covered the main aspects of AS technology and its enormous therapeutic potential (Anazodo et al. (1995) Gene 166(2):227-232). There are reviews on the chemical (Crooke S T (1995)Hematol Pathol. (Review) 9(2):59-72; Uhlmann et al.(2000) Methods Enzymol. 313:268-284.), cellular (Wagner R W (1994) Nature (Review) 372(6504):333-335), and therapeutic (Hanania et al. (1995) Am J. Med.(Review) 99(5):537-552; Scanlon et al. (1995) FASEB J. (Review) 9(13):1288-1296; Gewirtz A M (1993) Leuk Lymphoma. 1993; 11 Suppl 1:131-137) aspects of this rapidly developing technology. PCT publication WO 01/36646 (Glover et al).

RNA interference (siRNA or RNAi) technology may also be used in the methods of this invention. By “silencing RNA” (siRNA) is meant an RNA molecule which decreases or silences (prevents) the expression of a gene/mRNA of its endogenous or cellular counterpart. The term is understood to encompass “RNA interference” (RNAi), and “double-stranded RNA” (dsRNA). For information on these terms and proposed mechanisms, see Bernstein E., Denli A M., Hannon G J: The rest is silence. RNA. 2001 November; 7(11):1509-21; Nishikura K.: A short primer on RNAi: RNA-directed RNA polymerase acts as a key catalyst. Cell. 2001 Nov. 16; 107(4):415-8 and PCT publication WO 01/36646 (Glover et al). For disclosure on how to prepare siRNA to known genes see for example Chalk A M, Wahlestedt C, Sonnhammer E L. Improved and automated prediction of effective siRNA Biochem. Biophys. Res. Commun. 2004 June 18; 319(1):264-74; Sioud M, Leirdal M., Potential design rules and enzymatic synthesis of siRNAs, Methods Mol. Biol. 2004; 252:457-69; Levenkova N, Gu Q, Rux J J.: Gene specific siRNA selector Bioinformatics. 2004 Feb. 12; 20(3):430-2. and Ui-Tei K, Naito Y, Takahashi F, Haraguchi T, Ohki-Hamazaki H, Juni A, Ueda R, Saigo K., Guidelines for the selection of highly effective siRNA sequences for mammalian and chick RNA interference Nucleic Acids Res. 2004 Feb. 9; 32(3):936-48.5e also Liu Y, Braasch D A, Nulf C J, Corey D R. Efficient and isoform-selective inhibition of cellular gene expression by peptide nucleic acids Biochemistry, 2004 Feb. 24; 43(7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September; 9(9):1034-48 and U.S. Pat. Nos. 5,898,031 and 6,107,094 (Crooke) for production of modified/more stable siRNAs.

Delivery systems aimed specifically at the enhanced and improved delivery of siRNA into mammalian cells have been developed, see, for example, Shen et al (FEBS letters 539: 111-114 (2003)), Xia et al., Nature Biotechnology 20: 1006-1010 (2002), Reich et al., Molecular Vision 9: 210-216 (2003), Sorensen et al. (J. Mol. Biol. 327: 761-766 (2003), Lewis et al., Nature Genetics 32: 107-108 (2002) and Simeoni et al., Nucleic Acids Research 31, 11: 2717-2724 (2003). siRNA has recently been successfully used for inhibition in primates; for further details see Tolentino et al., Retina 24(1) February 2004 pp 132-138.

siRNA for HNOEL-iso can be made using methods known in the art as described above, based on the known sequence of HNOEL-iso (SEQ ID NO: 1), and can be made stable by various modifications as described above

As used herein, the term “polynucleotide” includes polynucleotides and oligonucleotides. Modifications or analogs of nucleotides can be introduced to improve the therapeutic properties of the polynucleotide. Improved properties include increased nuclease resistance and/or increased ability to permeate cell membranes.

Accordingly, the present invention also includes all analogs of, or modifications to, a polynucleotide of the invention that does not substantially affect the function of the polynucleotide.

The nucleotides to be modified can be selected from naturally occurring or synthetically modified bases. Naturally occurring bases include adenine, guanine, cytosine, thymine and uracil. Modified bases of the polynucleotides include xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structures of the nucleotides are fundamentally altered and are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone similar to that found in peptides. PNA analogs have been shown to be resistant to degradation by enzymes and to have extended lives in vivo and in vivo. Further, PNAs have been shown to bind more strongly to a complementary DNA sequence than to a DNA molecule. This observation is attributed to the lack of charge repulsion between the PNA strand and the DNA strand. Other modifications that can be made to polynucleotides include polymer backbones, cyclic backbones, or acyclic backbones.

The active ingredients of the pharmaceutical composition can include polynucleotides that are nuclease resistant, needed for the practice of the invention, or a fragment thereof shown to have the same effect targeted against the appropriate sequence(s) and/or ribozymes. Combinations of active ingredients as disclosed in the present invention can be used, including combinations of AS RNA or combinations of siRNA.

The AS polynucleotides, ribozymes, siRNA and cDNA of the present invention can be synthesized by any method known in the art for ribonucleic or deoxyribonucleic nucleotides. For example, an Applied Biosystems 380B DNA synthesizer can be used. When fragments are used, two or more such sequences can be synthesized and linked together for use in the present invention.

The polynucleotides of the present invention can be delivered either directly or with viral or non-viral vectors. When delivered directly the polynucleotides are generally rendered nuclease resistant. Alternatively the polynucleotide can be incorporated into and expression cassette or construct such that the polynucleotide is expressed in the cell as discussed herein below. Generally the construct contains the proper regulatory sequence or promoter to allow the polynucleotide to be expressed in the targeted cell.

The promoter exemplified is specific for the kidney. One skilled in the art can choose promoter specific to the heart, lungs, liver or any other organ of interest. Examples of such promoters include without limitation albumin and transthyretin promoters for liver hepatocytes, alpha myosin heavy chain promoter for heart and surfactant protein C or beta (2)-adrenergic receptors for lung.

The polypeptides of the present invention may be produced recombinantly (see generally Marshak et al., 1996 “Strategies for Protein Purification and Characterization. A laboratory course manual.” Plainview, N.Y.: Cold Spring Harbor Laboratory Press, 1996) and analogs may be produced by post-translational processing. Differences in glycosylation can provide polypeptide analogs.

As used herein, the term “polypeptide” refers to, in addition to a polypeptide, a peptide and a protein. As used herein, “biological functional” refers to the biological property of the molecule and in this context means an in vivo effector or antigenic function or activity that is directly or indirectly performed by a naturally occurring polypeptide or nucleic acid molecule. Biological functions include but are not limited to receptor binding, any enzymatic activity or enzyme modulatory activity, any carrier binding activity, any hormonal activity, any activity in internalizing molecules or translocation from one compartment to another, any activity in promoting or inhibiting adhesion of cells to extracellular matrix or cell surface molecules, or any structural role, as well as having the nucleic acid sequence encode functional protein and be expressible. The antigenic functions essentially mean the possession of an epitope or an antigenic site that is capable of cross-reacting with antibodies raised against a naturally occurring protein. Biologically active analogs share an effector function of the native polypeptide that may, but need not, in addition possess an antigenic function.

This application is also directed to a method of diagnosing fibrosis related pathologies in general, as detailed above and in particular this application is directed to a method of diagnosing liver fibrosis, pulmonary fibrosis and cardiac fibrosis.

Accordingly, the present invention provides a method of diagnosing a fibrosis in a subject comprising determining in a sample from the subject the level of HNOEL-iso polypeptide or the level of HNOEL-iso polypeptide-encoding polynucleotide, wherein a higher level of the polypeptide or the polynucleotide compared to the levels in a subject free of such fibrosis is indicative of such fibrosis. The sample in such method is taken from a bodily fluid, and more preferably from the group of fluids consisting of blood, lymph fluid, ascites, serous fluid, pleural effusion, sputum, cerebrospinal fluid, lacrimal fluid, synovial fluid, saliva, stool, sperm and urine.

Measurement of level of the HNOEL-iso polypeptide may be determined by a method selected from the group consisting of immunohistochemistry, western blotting, ELISA, antibody microarray hybridization and targeted molecular imaging. Such methods are well-known in the art, for example for immunohistochemistry: M. A. Hayat (2002) Microscopy, Immunohistochemistry and Antigen Retrieval Methods For Light and Electron Microscopy, Kluwer Academic Publishers; Brown C (1998): “Antigen retrieval methods for immunohistochemistry”, Toxicol Pathol; 26(6): 830-1); for Western blotting: Laemmeli UK (1970): “Cleavage of structural proteins during the assembly of the head of a bacteriophage T4”, Nature; 227: 680-685; and Egger & Bienz(1994) “Protein (western) blotting”, Mol Biotechnol; 1(3): 289-305); for ELISA: Onorato et al.(1998) “Immunohistochemical and ELISA assays for biomarkers of oxidative stress in aging and disease”, Ann NY Acad Sci 20; 854: 277-90); for antibody microarray hybridization: Huang(2001) “Detection of multiple proteins in an antibody-based protein microarray system, Immunol Methods 1; 255 (1-2): 1-13); and for targeted molecular imaging: Thomas (2001). Targeted Molecular Imaging in Oncology, Kim et al (Eds)., Springer Verlag, inter alia.

Measurement of level of HNOEL-iso polynucleotide may be determined by a method selected from: RT-PCR analysis, in-situ hybridization, polynucleotide microarray and Northern blotting. Such methods are well-known in the art, for example for in-situ hybridization Andreeff & Pinkel (Editors) (1999), “Introduction to Fluorescence In Situ Hybridization: Principles and Clinical Applications”, John Wiley & Sons Inc.; and for Northern blotting Trayhurn (1996) “Northern blotting”, Proc Nutr Soc; 55(1B): 583-9 and Shifman & Stein (1995) “A reliable and sensitive method for non-radioactive Northern blot analysis of nerve growth factor mRNA from brain tissues”, Journal of Neuroscience Methods; 59: 205-208 inter alia.

Measurement of effect of the compound on a parameter related to fibrosis and comparing the effect measured with the effect measured in the absence of the compound may be determined by any of the methods described in the examples of the present invention or by any method known to one skilled in the art.

Another embodiment of the invention provides a method for diagnosis of fibrosis in a body fluid sample from a subject comprising:
(i) contacting the sample with an antibody specific to HNOEL-iso polypeptide under conditions enabling the formation of an antibody-antigen complex;
(ii) determining the level of antibody-antigen complex formed, wherein a determination of the presence of a level of antibody-antigen complex significantly higher than that formed in a control sample indicates fibrosis in the subject.

In one embodiment of the invention, a method is provided for the treatment of fibrosis and fibrosis related pathologies in a subject in need of such treatment comprising administering to said subject an amount of an inhibitor of HNOEL-iso polypeptide sufficient to effect a substantial inhibition of the HNOEL-iso polypeptide so as to thereby treat the subject. By “substantial inhibition” of the HNOEL-iso polypeptide is meant inhibition to between 0-50%, preferably to between 0 to 30%, more preferably to between 0-15% or most preferably to 0-5% of the HNOEL-iso polypeptide level before treatment. In particular embodiments the inhibitor is an antibody, siRNA or antisense RNA and the fibrosis is selected from liver fibrosis, pulmonary fibrosis and cardiac fibrosis.

### METHODS

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.)

### General Methods in Immunology

Standard methods in immunology known in the art and not specifically described are generally followed as in Stites et al (eds), Basic and Clinical Immunology (8th Edition), Appleton & Lange, Norwalk, Conn. (1994) and Mishell and Shiigi (eds), Selected Methods in Cellular Immunology, W. H. Freeman and Co., New York (1980).

### Immunoassays

In general ELISAs, where appropriate, are one type of immunoassay employed to assess a specimen. ELISA assays are well known to those skilled in the art. Both polyclonal and monoclonal antibodies can be used in the assays. Where appropriate other immunoassays, such as radioimmunoassays (RIA) can be used as are known to those skilled in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al., Molecular Cloning: A Laboratory Manual, Cold Springs Harbor, N.Y., 1989.

### Antibody Production

By the term “antibody” as used in the present invention is meant both poly- and mono-clonal complete antibodies as well as fragments thereof, such as Fab, F(ab′)2, and Fv, which are capable of binding the epitopic determinant. These antibody fragments retain the ability to selectively bind with its antigen or receptor and are exemplified as follows, inter alia:
(1) Fab, the fragment which contains a monovalent antigen-binding fragment of an antibody molecule can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′2) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

Such fragments having antibody functional activity can be prepared by methods known to those skilled in the art (Bird et al. (1988) Science 242:423-426)

Conveniently, antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody (see generally Huston et al. (1991) “Protein engineering of single-chain Fv analogs and fusion proteins” in Methods in Enzymology (J J Langone, ed., Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Memaugh and Mernaugh (1995) “An overview of phage-displayed recombinant antibodies” in Molecular Methods In Plant Pathology (R P Singh and U S Singh, eds.; CRC Press Inc., Boca Raton, Fla.:359-365), messenger RNAs from antibody-producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. For a general discussion of conjugation of fluorescent or enzymatic moieties see Johnstone & Thorpe (1982.), Immunochemistry in Practice, Blackwell Scientific Publications, Oxford. The binding of antibodies to a solid support substrate is also well known in the art; for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co. The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

### Recombinant Protein Purification

For standard purification, See Marshak et al. (1996), “Strategies for Protein Purification and Characterization. A laboratory course manual.” CSHL Press. Specific purification protocols used for the production of HNOEL-iso protein are described in the Examples below.

### Transgenic and Knockout Methods

The present invention provides for a transgenic gene and a polymorphic gene animal and cellular (cell line) model, as well as for a knockout model. These models are constructed using standard methods known in the art and as set forth in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; 4,736,866; as well as Burke and Olson (1991) “Preparation of Clone Libraries in Yeast Artificial-Chromosome Vectors” in Methods in Enzymology, 194, “Guide to Yeast Genetics and Molecular Biology”, eds. C. Guthrie and G. Fink, Academic Press, Inc., Chap. 17:251-270; Capecchi (1989) “Altering the genome by homologous recombination”, Science, 244:1288-1292; Davies et al. (1992) “Targeted alterations in yeast artificial chromosomes for inter-species gene transfer”, Nucleic Acids Research, 20 (11): 2693-2698; Dickinson et al. (1993) “High frequency gene targeting using insertional vectors”, Human Molecular Genetics, 2(8):1299-1302; Duff and Lincoln (1995) “Insertion of a pathogenic mutation into a yeast artificial chromosome containing the human APP gene and expression in ES cells”, Research Advances in Alzheimer's Disease and Related Disorders Khalid Iqbal (Editor), James A. Mortimer (Editor), Bengt Winblad (Editor), Henry M. Wisniewski (Editor); Huxley et al. (1991) “The human HPRT gene on a yeast artificial chromosome is functional when transferred to mouse cells by cell fusion”, Genomics, 9:742-750; Jakobovits et al. (1993) “Germ-line transmission and expression of a human-derived yeast artificial chromosome”, Nature, 362: 255-261; Lamb et al. (1993) “Introduction and expression of the 400 kilobase precursor amyloid protein gene in transgenic mice”, Nature Genetics, 5:22-29; Pearson and Choi (1993) Expression of the human b-amyloid precursor protein gene from a yeast artificial chromosome in transgenic mice. Proc. Natl. Acad. Sci. (USA), 90:10578-10582; Rothstein, (1991) “Targeting, disruption, replacement, and allele rescue: integrative DNA transformation in yeast” in Methods in Enzymology, 194, “Guide to Yeast Genetics and Molecular Biology”, eds. C. Guthrie and G. Fink, Academic Press, Inc., NY, Chap. 19:281-301; Schedl et al. (1993) “A yeast artificial chromosome covering the tyrosinase gene confers copy number-dependent expression in transgenic mice”, Nature, 362:258-261; Strauss et al. (1993) “Germ line transmission of a yeast artificial chromosome spanning the murine a, (I) collagen locus”, Science, 259:1904-1907. Further, PCT patent applications WO 94/23049, WO 93/14200, WO 94/06908, WO 94/28123 also provide information.

Further one parent strain, instead of carrying a direct human transgene, may have the homologous endogenous gene modified by gene targeting such that it approximates the transgene. That is, the endogenous gene has been “humanized” and/or mutated (Reaume et al. (1996) J Biol. Chem. 271(38):23380-23388.). It should be noted that if the animal and human sequences are essentially homologous, a “humanized” gene is not required. The transgenic parent can also carry an overexpressed sequence, either the non-mutant or a mutant sequence and humanized or not as required. Herein, the term “transgene” is therefore used to refer to all these possibilities. Additionally, cells can be isolated from the offspring that carry a transgene from each transgenic parent and that are used to establish primary cell cultures or cell lines as is known in the art.

Where appropriate, a parent strain will be homozygous for the transgene. Additionally, where appropriate, the endogenous non-transgene in the genome that is homologous to the transgene will be non-expressive. Herein, by the term “non-expressive” is meant that the endogenous gene will not be expressed and that this non-expression is heritable in the offspring. For example, the endogenous homologous gene could be “knocked-out” by methods known in the art. Alternatively, the parental strain that receives one of the transgenes could carry a mutation at the endogenous homologous gene rendering it non-expressed.

The present invention is illustrated in detail below with reference to Examples, but is not to be construed as being limited thereto.

### EXAMPLES

### Example 1

### Identification of HNOEL-Iso Overexpression by Microarray Hybridization Study

In accordance with the present invention, the microarray hybridization approach was utilized in order to discover genes that are differentially regulated in diabetic nephropathy and kidney fibrosis. Microarray-based analysis of gene expression was based on the analysis of human fibroblasts subject to selected stimuli resulting in changes in extracellular collagen accumulation and proliferation—the hallmarks of fibrosis. According to the present invention, a specific “Fibrosis” DNA chip was first prepared followed by a microarray hybridization experiments with 19 different types of probes. Analysis of the results was carried out by proprietary algorithms, and analysis of the selected set of genes was performed by the inventors using bioinformatics and the scientific literature.

Preparation of specific “Fibrosis” DNA Chip

A dedicated human “Fibrosis” DNA chip was prepared according to the co-assigned patent application which is directed to the SDGI method (PCT Application Publication No. WO 01/75180) from growth-arrested human fibroblasts. Growth arrest was imposed by the treatments presented in Table 1 below:

TABLE 1
Biological material for “Fibrosis” chip preparation

| | Treatment |
|---|---|
| 1 | G1 arrested serum-starved l.p. HFs* |
| 2 | l.p. HFs* 36 hr and 48 hr following 8Gy γ-irradiation |
| 3 | l.p. HFs* 5 days after addition of H₂0₂ 200 μM |
| 4 | l.p. HFs* following UV (growth-arresting dose) |
| 5 | l.p. HFs* 48 hr following Bleomycin treatment 50 ng/ml |
| 6 | l.p. HFs* 48 hr following Etoposide treatment 400 ng/ml |
| 7 | l.p. HFs* 48 hr following Adriamycin treatment |
| | 50 ng/ml |
| 8 | Senescent HFs from normal individuals |
| 9 | Senescent HFs from individuals with Werner syndrome |
| 10 | Senescent HFs from individuals with Progeria |

l.p. HF*—low passage human fibroblasts

Unless indicated otherwise, all human fibroblasts (HFs) were at passage 15 prior to treatment. RNA from all treated HFs was prepared, pooled and used for library preparation by the proprietary SDGI method of the assignee. This chip also contained human ESTs coding for genes known to play a part in apoptosis, cytotoxicity and replicative cellular senescence.

### Fibroblast Cultivation

Normal human fetal lung fibroblasts (WI-38, Coriell Cell Repositories) were cultured and sub-cultured in DMEM, supplemented with 10% inactivated fetal bovine serum (FBS), 2 mM L-glutamine, 100 U/ml penicillin, 100 μg/ml streptomycin. Fibroblasts were grown to confluence in 25 cm² tissue flasks and sub-cultured after trypsinization (0.5% trypsin-EDTA in Hank's balanced solution without Ca²⁺ and Mg²⁺) at 37° C. in an atmosphere of 5% CO₂. Two ml of trypsin were added to each flask and incubated for 5 min; then cultures were centrifuged (5 min, 1000 rpm) and fresh medium was added to the pellet. Splitting conditions were 1:4-1:6.

Since the hallmarks of fibrotic disease are fibroblast proliferation and/or enhanced synthesis of extracellular matrix components (mainly collagen), different treatment regimes were used and the rates of both proliferation and collagen synthesis by the treated fibroblasts cultured in vitro was examined.

### Fibroblast Proliferation Assay

The proliferation rate of sub-confluent fibroblasts was evaluated by staining with neutral red (BioRad). Fibroblasts were seeded in 96-well plate (6×10³/well) in 200 μl of supplemented DMEM/10% FBS. After overnight culture, wells were washed twice with supplemented DMEM/2% FBS. Then, either TGF-β (2-20 ng/ml) or deferoxamine mesylate (DFO, which leads to conditions of chemical hypoxia) at a concentration of 100 mM was added in 200 μl of supplemented DMEM/2% FBS for either 16 hours, 24 hours, 72 hours, or 5 days.

In the case of glucose treatments, after overnight culture, cell-containing wells were washed twice with supplemented glucose-free DMEM/2% FBS. Working concentrations of glucose (5.5 mM, 15 mM, 27.5 mM, or 55 mM) were prepared by dissolving stock solution (110 mM) in supplemented DMEM without glucose/2% FBS. Prepared solutions of glucose were added to fibroblast cultures for either 24 or 72 hours.

Upon completion of incubation, cells were stained with 100 μl of 1% neutral red for 2 hours. After washing with cold PBS, fibroblast monolayers were fixed with 200 μl of ethanol-Sorenson buffer solution (1:1) for 10 minutes. Optical density was measured with an automated spectrophotometer (λ=540 nm).

### Collagen Production Assay

Collagen production by confluent fibroblast monolayers was assessed by [³H]-proline incorporation into collagenous proteins. Fibroblasts were seeded in 24-well tissue culture plates (2×10⁴/well) and grown in 1 ml of supplemented DMEM/10% FBS until confluence. Confluent fibroblast cultures were incubated with prepared solutions for either 24 or 48 hours. Then [³H]-proline (10 μCi/well) was added and cultures were incubated for an additional 24 hours. At the end of the incubation, medium was decanted and incubated with or without collagenase for 18 hours, followed by precipitation with 50% and 10% TCA. The production of collagen was determined as the difference between total [³H]proline-containing proteins in the sample incubated without collagenase and those left after collagenase digestion. To determine the number of cells in each well, fibroblasts were detached by trypsinization on the last day of the experiment, and counted in a hemocytometer.

Probes for microarray hybridization were derived from these treated fibroblasts. In accordance with the present invention, treatments that are relevant for diabetic nephropathy development were used, such as glucose deprivation or hypoxia (modeling ischemic conditions that develop in fibrotic kidney); high glucose (modeling diabetic hyperglycemia) and TGF-β induction (modeling a fibrotic condition that is characterized by growth factor and cytokine imbalance).

More specifically, human fibroblasts were treated as followed:
1. glucose at 4 different concentrations (5.5, 15, 27.5, or 55 mM) for 24 and 72 hours
2. TGF-β at 2-20 ng/ml, for 24 or 72 hours
3. DFO deferoxamine at a concentration of 100 mM, dissolved in 0.5 ml of DMEM, containing 5% FCS, 50 μg/ml β-aminoproprionitrile, and 50 μg/ml ascorbic acid (modified DMEM), for 24, 48 and 72 hours.

The analysis of proliferation rate of these cultured fibroblasts showed that cultivation of fibroblasts for 24 hrs in glucose-free medium and in 55 mM glucose resulted in a decrease of their proliferation rate by 20% and 30%, respectively, compared to control cultures. Addition of glucose at different concentrations (from 5.5 mM to 27.5 mM) virtually did not affect fibroblast proliferation compared to the control. A significant decrease in fibroblast proliferation was observed after addition of DFO (from 20% decrease after 16 hours incubation to 80% decrease after 5 days of treatment). TGF-β, added at concentrations of 2 and 20 ng/ml, led to an increase in the fibroblast proliferation rate by ˜60% after 24 hours treatment.

As for collagen synthesis rate, all treatments (except for 55 mM glucose) led to increased collagen production by fibroblasts. The most significant effect was observed after addition of TGF-β concentrations of 2-20 ng/ml, providing enhancement in collagen production by 110-180%.

In the next step, the RNA from these treated fibroblasts was extracted and used for preparation of probes for microarray hybridization. The scheme of hybridization is presented below:

TABLE 2
Hybridization scheme

| PROBE | | | Probe | | |
|---|---|---|---|---|---|
| NAME | DYE | PROBE 1 | name | DYE | PROBE 2 |
| FG1A | Cy3 | Untreated human fibroblasts- | FG1B | Cy5 | 1.p. untreated HFs* |
| FG19A | | Common Normalizing Probe | FG19B | | 1.p. untreated HFs* |
| FG18A | | | FG18B | | 1.p. HFs* w/o glucose 72 hr |
| FG17A | | | FG17B | | 1.p. HFs* TGF-β 20 ng/μl72 h |
| FG16A | | | FG16B | | 1.p. HFs* TGF-β 20 ng/μl 24 h |
| FG15A | | | FG15B | | 1.p. HFs* w/o glucose 24 h |
| FG14A | | | FG14B | | 1.p. HFs* TGF-β 2 ng/μl 72 h |
| FG13A | | | FG13B | | 1.p. HFs* TGF-β 2 ng/ml 24 h |
| FG12A | | | FG12B | | 1.p. HFs* 5.5 mM glucose 72 h |
| FG11A | | | FG11B | | 1.p. HFs* 5.5 mM glucose 24 h |
| FG10A | | | FG10B | | 1.p. HFs* Hypoxia 5 days |
| FG9A | | | FG9B | | 1.p. HFs* 55 mM glucose 72 h |
| FG8A | | | FG8B | | 1.p. HFs* 55 mM Glucose 24 h |
| FG7A | | | FG7B | | 1.p. HFs* Hypoxia 3 days |
| FG6A | | | FG6B | | 1.p. HFs* 27.5 mM Glucose |
| | | | | | 72 h |
| FG5A | | | FG5B | | 1.p. HFs* 27.5 mM glucose |
| | | | | | 24 h |
| FG4A | | | FG4B | | 1.p. HFs* hypoxia 16 h |
| FG3A | | | FG3B | | 1.p. HFs* 15 mM glucose 72 h |
| FG2A | | | FG2B | | 1.p. HFs* 15 mM glucose 24 h |

1.p. HFs* - low passage human fibroblasts

Probe 1 was identical in all hybridization experiments, and was produced with RNA extracted from untreated human fibroblasts (passage 15). This probe served both as a biological control and as a common normalizing probe that allowed comparison of results obtained from different hybridization experiments.

In accordance with the present invention, a total of 19 hybridization experiments were performed. In two hybridization experiments (FG1 and FG19), the common normalizing probe (Probe 1 in all hybridization experiments) was hybridized against itself (i.e., Probe 1 was identical to Probe 2). In general, these hybridization experiments were conducted in order to determine labeling quality and to evaluate the ability of the common normalizing probe to detect most of the cDNA clones printed on the chip.

### Bioinformatics Analysis of Gene Expression Results

The proprietary statistical algorithm of the assignee was used to analyse the microarray hybridization results, based on the assumption that changes in gene expression correlate with different physiological and pathological conditions and, in many instances, underlie them. Thus, in a given set of experiments, a certain treatment regime/condition is associated with a particular gene expression profile. Furthermore, the inventors assumed that some hierarchy exists among the different pathological conditions/physiological treatments, i.e., some are more similar than others.

The final goal of such an analysis was to elucidate both specific and general mechanisms underlying complex biological phenomena by comparison of gene expression patterns within a large panel of conditions, each representing some of its aspects. More specifically, in the set of hybridization results generated in accordance with the present invention, the inventors anticipated observing groups of genes the expression of which was either common or unique to different types of conditions relevant to diabetic nephropathy (hypoxia, high glucose, TGF-β, and wherein the response to the applied treatment was either acute or chronic.

### Results of Hybridization Analysis

In accordance with the present invention, in human fibroblasts differentially treated in vitro, a set of 46 genes was identified, the activity of which was significantly up-regulated by various types of applied treatments.

The identified gene products fell into nine distinct functional groups:
1. Extracellular matrix proteins and receptors to extracellular matrix proteins;
2. Secreted growth factor interacting proteins and potential growth factor receptors;
3. Signal transduction adaptor proteins;
4. Cytoskeletal proteins (mostly related to actin cytoskeleton function);
5. Ca²⁺-binding proteins;
6. ER-resident proteins;
7. Nuclear import mediators;
8. Proteins involved in RNA and protein synthesis and processing;
9. Novel genes;

The 46 up-regulated genes identified were divided as follows:
(a) 11 were known genes with known functions with recognized involvement in fibrosis (collagens type III and I (α1 and α2), fibronectin, decorin, β-ig-h3, integrin, TIMP3, CD44, smooth muscle actin, and Arp2/3 (Arc34);
(b) 28 were known genes with known function but with previously unknown involvement in fibrosis;
(c) 2 were genes coding for proteins with unknown function and unknown involvement in fibrosis. HNOEL-iso, the subject of the present application, falls into this category;
(d) 5 were novel genes.

Using the microarray hybridisation technique it was found that the expression of HNOEL-iso was induced by TGF-β treatment of human fibroblasts by at least 2-3 fold.

### Example 2

### Construction of Kidney Specific Promoter (KSP) for Transgenic Mice

The KSP-cadherin gene promoter (3593 bp) which is known to be tubular specific (epithelial cell specific) promoter was cloned in pMCSZ vector which contains lacZ. Transgenic expression of lacZ reporter gene controlled by kidney-specific cadherin promoter was evaluated in transient transgenic mouse embryos. One out of 9 E18.5 embryos and two out of 8 E15.5 embryos showed a specific expression pattern in the kidneys (the expression in the E15.5 kidneys was much weaker). The expression was located towards the medullary region, in the center of the metanephros (a wholemount staining).

Analysis of sections of wholemount stained kidneys of the E18.5 embryo revealed the transgenic expression in tubular epithelial cells, which according to their location and characteristic branching seem to be the collecting ducts. Expression was also evident throughout the ureter. The collecting ducts develop from the branching ureteric bud, and at this stage of development, the majority of the collecting duct system is contained in the medullary region of the metanephros. No expression was observed in the subcapsular nephrogenic zone.

These results are in correlation with those described by Igarashi et al. 1999 (Am. J. Physiol. 277 (4 pt 2). By co-labeling the lacZ expressing cells with Dolichos biflorus agglutinin, a lectin that specifically labels the collecting ducts and uretheric buds, they have identified the lacZ expressing cells as epithelial tubular cells. They have also reported that the expression of the transgene increases during gestation (similar to our findings) and remains high in the adult kidney

In conclusion, we find that the KSP-Cadherin promoter is a specific promoter for kidney epithelial cell expression. No expression of the lacZ gene was obtained in any other tissue except the kidney.

### Example 3

### Establishment of Transgenic Mice Expressing the HNOEL-Iso in Mouse Kidneys

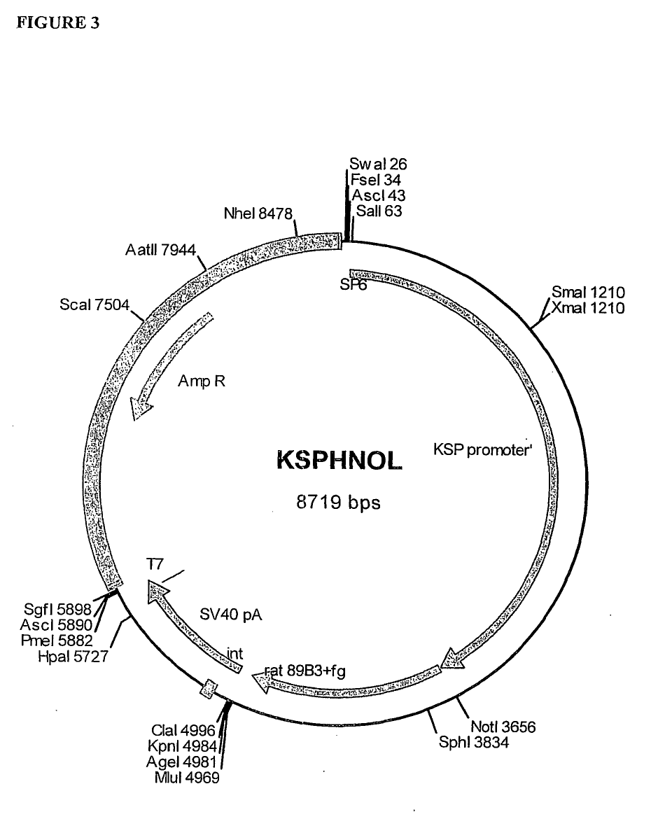

The rat HNOEL gene (r89B7F) was inserted into the Not1/Mlu1 sites of the KSPMCS vector, containing the KSP-cadherin gene promoter (3593 bp) which is known to be tubular specific (epithelial cells specific) promoter (FIG. 3). The Asc-1 fragment (5500 bp) containing the KSP promoter, the gene and the SV40 region was cut from the plasmid and injected into FVBN mouse eggs.

87 of the newborns were checked for transgenic integration. Out of the 87 pups born 9 were found to carry the HNOEL gene. These were further analysed for the expression of HNOEL-ISO by Northern analysis. RNA from 4-week-old animals was prepared using EZ-RNA total RNA isolation kit (Biological Industries). Either an SV40 probe (exogenous) or an HNOEL specific probe were used for this analysis. 6 of the 9 lines were found to express the gene in high levels. 3 of these lines, designated 80 female, 79 female & 5 male, that showed the highest expression of the transgene were further analyzed by in-situ analysis (see Example 4).

### Example 4

### In Situ Hybridization Analysis on KSP-HNOEL-Iso Transgenes

In situ hybridization analysis was performed on KSP-HNOEL mice. Six mice, from three different transgenic lines—designated 80 female, 79 female & 5 male—and two nontransgenic control mice were analyzed.

Age of animals: 4-6 weeks;

Probes used: HNOEL and SV-40 probe which recognizes transgenic expression only. GAPDH probe was used as a tissue control.

Results showed that all animals (transgenic and wild type) exhibited a normal structure of kidney tissue.

Expression of endogenous HNOEL in kidneys could not be detected in the nontransgenic control mice.

A high level of exogenous expression appeared in all three transgenic lines, in agreement with Northern blot results. The signal was localized mainly in the collecting ducts, both in the medulla and cortical regions.

### Example 5

### Protein Analysis on HNOEL-iso in Transgenic Kidney

Western blot analysis was done to evaluate the protein expression level of HNOEL-iso in transgenic kidneys. The expected protein size was 64 kDa. Cells transiently transfected with HNOEL-ISO served as control. The detection of HNOEL was carried out by both a specific polyclonal antibody raised against a peptide from HNOEL-iso (in transgenic and wild type animals) and with a flag antibodies.

The results showed the existence of a band of the expected size in both control cells in the mouse kidneys from lines 80, 79 and 5. The amount of protein detected in the kidneys from transgenic animals was much higher then its level in wild type animals.

### Example 6

### Assessment of In Vivo Models for Kidney Fibrosis by Morphology, Immunostaining and In Situ Hybridization

### Morphology

To assess general morphology, paraffin kidney sections were stained by hematoxilin-eosin (HE). Sirius Red (SR) staining was used to reveal collagen in the sections.

### Immunostaining

Accumulation of interstitial myofibroblasts is regarded as an important initial step in the development of the renal fibrotic process. To reveal myofibroblasts, monoclonal antibody specific to α-smooth muscle actin (clone 1A4) was used for the peroxidase-antiperoxidase (PAP) immunostaining of kidney paraffin sections. The monoclonal antibody PC-10 was used for the immunostaining of proliferating cell nuclear antigen (PCNA). To achieve adequate PCNA immunostaining, de-paraffinized sections were subjected to antigen retrieval procedure before performing PAP staining.

### In Situ Hybridization

³⁵S-labeled riboprobes were synthesized and hybridized to kidney paraffin sections according to standard protocol. After the post-hybridization washing step, sections were air-dried and macro-autoradiography was performed by exposing the slides to X-ray film overnight. For micro-autoradiography, slides were dipped into nuclear track emulsion and stored in darkness at 4° C. Exposed slides were developed after 2-3 weeks and sections were slightly counter-stained with HE and cover-slipped for microscopic examination.

### Probes for In Situ Hybridization

The cDNAs used as the templates for riboprobe synthesis were rat osteopontin cDNA, mouse transforming growth factor β1 cDNA, mouse procollagen α1(I) cDNA and mouse thrombospondinl cDNA.

### Examples of Models

### ZDF Rats

Samples of 9-month-old ZDF rats (Zucker diabetic fatty rats) presented hydronephrotic kidneys with dilated calyces. Microscopically these samples presented a picture of glomerulosclerosis and tubulointerstitial fibrosis. In accordance with these morphological changes, the expression of marker genes as measured by in situ hybridization (osteopontin (OPN), transforming growth factor β1 (TGF-β1) and procollagen α1(I) (Col1)) was significantly changed when compared to normal kidneys. Strong OPN expression was detectable in all tubular structures in both cortex and medulla. The TGF-β1 expression was widespread throughout interstitial cells. Some epithelial cells also showed TGF-β1 expression. Col1 expression was detectable by in situ hybridization in most interstitial cells within the medulla, while cortical expression was “focal”.

Aged fa/fa (Obese Zucker) Rats

Samples of 12-month-old fa/fa rats presented strong glomerulosclerosis and diffuse tubulointerstitial fibrosis throughout the cortex and the medulla. The pattern of marker gene expression corresponded to morphological changes. OPN was expressed by tubular structures in the cortex and the medulla. Multiple interstitial cells expressed TGF-β1. Significantly, multiple foci and single interstitial cells showed strong Col1 expression in both cortex and medulla so that the number of Col1-expressing cells appeared to be higher in fa/fa samples than in ZDF samples.

Interestingly, Col1 expression was not detected in glomeruli of either ZDF or fa/fa rats in spite of the prominent accumulation of collagen, as revealed by Sirius Red staining. This suggested a low steady state level of Col1 mRNA in glomerular cells

### Aged SD (Normal) Rats

Samples of aged SD rats showed increased accumulation of collagen in glomeruli and interstitial space and increased expression of the marker genes. Significantly, the intensity of fibrotic change varied among samples so that one of four samples studied displayed very few changes compared with young animals; fibrotic change in another sample was confined to “polar” regions, and two samples showed uniform accumulation of collagen and elevated expression of marker genes throughout the sections.

### Goto Kakizaki (GK)/Wistar (Normal) 48-Week-Old Rats

Samples of both GK and Wistar 48-week-old rats showed an accumulation of collagen in glomeruli and interstitial space. This accumulation was more pronounced in the GK samples. Two samples were used for mRNA isolation: C9 and GK9. Both were hybridized to the probe specific for IGFBP4. The in situ hybridization results showed that the GK sample demonstrated elevated expression of this gene.

### Permanent UUO

A known model for fibrosis was employed which is unilateral ureter occlusion (UUO) One of the ureters was occluded (see below) and animals were sacrificed 1,5,10,15,20 and 25 days following occlusion.

Permanent UUO resulted in rapid activation (5 days of UUO) of collagen synthesis by interstitial cells in both medulla and cortex. By 20-25 days of UUO, significant amounts of interstitial collagen were deposited in the interstitial space while glomerular accumulation of collagen was confined to the outer capsule. Thus, permanent UUO samples provided an acute model of tubulointerstitial kidney fibrosis without prominent glomerulosclerotic changes.

The above models can be used as model systems for testing the therapeutic efficacy of modulators of HNOEL-ISO identified via any of the screening systems described.

### Example 7

### Protocol for Permanent Unilateral Ureteral Obstruction in Rats(UUO)

### Test System

Strain: Male Sprague-Dawley rats (9 weeks of age)

Group Size: n=5 for operated rat; n=3 for sham-operated rats

Number of groups: 6 for both sham-operated and operated (i.e., 1 day, 5 days, 10 days, 15 days, 20 days and 25 days post-operation or post-sham operation)

### Procedure

Rats were anaesthetized with Ketamin/Xylazine and the abdominal cavity was opened. After being exposed, the ureter from the right kidney was ligated with a suture over it (UUO). In sham-operated rats, the ureter was exposed but not ligated.

### Study Termination

The study was terminated 24 hr, 5 days, 10 days, 15 days, 20 days and 25 days after the UUO procedure or after the sham operation. At this time point, the rats were sacrificed by exsanguination under CO₂ asphyxiation in order to collect the right kidney. After the capsule was removed the kidney was cut transversely. Half was fixed in 10% buffered formalin and the other half was immediately transferred to an eppendorf tube and frozen in liquid nitrogen for RNA analysis.

### Example 8

### Evaluation of fibrosis after Permanent Unilateral Uretel Obstruction (UUO) in ksp-HNOEL Transgenic Mice

To investigate the development of kidney fibrosis in the presence of HNOEL over-expression, unilateral ureteral obstruction (UUO) was performed in transgenic (TG) mice of ksp-HNOEL. Total of 52 male mice, FVB-N TG for ksp-HNOEL and their wild type (WT) littermates were analysed at 6-10 weeks of age (body weight 20-30 g) after UUO operation. The Permanent Unilateral Ureter Obstruction was performed using methods known in the art as described below:

Medial regional abdominal dissection was performed via the Linea Alba in all mice. Following Equithesine IP anesthesia, an atraumatic intestine displacement was performed to allow the exposure of the left ureter. The exposed ureter was then double ligated using 7-0 sterile blue virgin silk The upper ligation was consistently placed exactly at the level of the lower kidney pole. The intestine was returned to its normal anatomic position. Thereafter, the abdominal muscles and skin was hermetically closed in layers using 4-0 (or 5-0) silk. The right kidney was not ligated. Following the termination of the surgical procedure the animals received a single subcutaneous administration of an analgesic.

TABLE 3
Study groups and termination points:

| Time points for sacrificing of Operated | |
|---|---|
| groups | Number of animals |
| 3 days post operation | WT littermates - 7 |
| | TG ksp-HNOEL - 4 |
| 7 days post operation | WT littermates - 11 |
| | TG ksp-HNOEL - 10 |
| 14 days post operation | WT littermates - 10 |
| | TG ksp-HNOEL - 10 |

At each termination point the animals were sacrificed by CO₂ exposure and cervical dislocation. Both kidneys were excised and fixed in buffered formalin and processed as described in Example 10.

### Evaluation of Fibrosis

To evaluate the degree of fibrosis the amount of total collagen and the protein level per each kidney were measured using the SRFG protocol as described in detail in Example 10. Relative collagen content was calculated as (collagen level)/(protein level). Three independent measurements of collagen level and protein level were taken for each sample (Table 4). Also, the total kidney volume (see Example 12 for protocol) was compared between the two types of animals (Table 5). For the 7 day time point, volume fraction of cortical interstitial tissue in obstructed kidneys was estimated (see Table 6 and Example 11). Statistical analysis of results was performed using Two Way Nested ANOVA. A summary of the results is provided in the tables below:

TABLE 4
Collagen content in kidney (microgram collagen/mg protein)

| | Contralateral kidney | | UUO treated kidney | |
|---|---|---|---|---|
| Days after | TG ksp- | WT | TG ksp- | WT |
| UUO | HNOEL* | littermates* | HNOEL * | littermates* |
| 3 | 8.63 | 7.86 | 10.56 | 8.60 |
| | (0.35) | (0.42) | (0.37) | (0.84) |
| 7 | 9.13 | 7.97 | 13.49 | 10.35 |
| | (0.74) | (0.41) | (1.19) | (0.69) |
| 14 | 9.52 | 7.89 | 15.24 | 13.31 |
| | (0.74) | (0.49) | (3.13) | (2.06) |

The mean of 3 measurements and standard deviation (in brackets) are provided.

TABLE 5
kidney volume (mm³)

| | Contrateral kidney | | UUO treated kidney | |
|---|---|---|---|---|
| Days after | TG ksp- | WT | TG ksp- | WT |
| UUO | HNOEL* | littermates* | HNOEL* | littermates* |
| 3 | 135.75 | 121.13 | 154.75 | 173.57 |
| | (19.95) | (22.54) | (14.29) | (34.73) |
| 7 | 140.00 | 127.45 | 119.49 | 153.09 |
| | (28.22) | (18.88) | (16.249) | (23.12) |
| 14 | 118.01 | 125.02 | 79.69 | 101.34 |
| | (18.18) | (14.83) | (17.16) | (10.55) |

*The mean of 3 measurements and standard deviation (in brackets) are provided.

TABLE 6
Interstitial volume fraction (%)

UUO treated kidney

| Days after UUO | TG ksp-HNOEL | WT littermates |
|---|---|---|
| 7 | 44.517 (SD-2.97) | 33.74 (SD-4.44) |

Relative collagen content was significantly higher in contralateral and obstructed kidneys of TG mice comparing to WT littermates. Total collagen content was higher in non-operated kidneys of TG mice while obstructed kidneys of both groups contained similar amount of total collagen.

Total protein content and total volume of kidney were lower in obstructed kidneys of TG mice than of WT mice.

Volume fraction of cortical interstitium was significantly higher in TG obstructed kidneys than in WT mice at 7 days of UUO.

These results suggest that over-expression of H-NOEL in kidneys of TG mice results in spontaneous kidney fibrosis, and that significant augmentation of tubulointerstitial fibrosis and renal dystrophy is stimulated by UUO. Thus, H-NOEL appears to be an important profibrotic factor, and inhibition of its expression/activity will be beneficial for the attenuation of fibrotic diseases.

### Example 10

### Renal Tissue Fixation, Processing and Collagen Content Determination (SRFG Protocol)

A procedure for systematic random sectioning of formalin fixed renal tissue was developed. The aim of the procedure was the exhaustive sectioning of the whole kidney and systematic collection of the representative sections for the histological staining.

Following removal of the kidneys, the capsule and surrounding tissues were removed. The ureter and external blood vessels at the entrance to the renal pelvis were also removed and the kidney was fixed with buffered formalin for 14-16 hrs. Fixed tissue was processed for paraffin embedding. Whole kidney was then cut into longitudinal 5 μm sections. A series of sequential sections separated by 500 μm was collected onto slides. This procedure results in collection of set of 10-12 sections representing whole kidney. For each sample three parallel sets of systematic sections were collected allowing performing triplicate SRFG staining.

Some arbitrary sections through the central part of the kidney (at level of papilla) were collected for additional histological analysis (see Example 11).

The collagen content determination was performed as described by Lopez-De Leon A, Rojkind M. 1985, “A simple micromethod for collagen and total protein determination in formalin-fixed paraffin-embedded sections”, J Histochem Cytochem. 33(8):737-43.

Calculation of the collagen content was performed according to published coefficients.

Corrected OD529 (CorrOD540) is calculated as:

CorrOD529=OD529−0.26×OD604

Total collagen (ColI) in mg is calculated as:

$$Coll = \frac{CorrOD 529}{37.8}$$

Total protein (Prot) in mg is calculated as:

$$Prot = \frac{O D 604}{2.04} + Coll$$

Collagen content (CollC) in μg of collagen per mg of total protein is calculated as:

$$CollC = \frac{1000 \times Coll}{Prot}$$

### Example 11

### Interstitial Volume Fraction Estimation

The following procedure has been developed for the assessment of the interstitial tissue volume fraction (Vi) in cortical part of kidney subjected to UUO. This estimation is performed on the arbitrary sections collected during the systematic sectioning of sections destined for the SRFG staining (see example 10).

Microphotography is performed using Zeiss Axioscope 2 microscope equipped with digital camera “Spot-2” providing color image comprised of 1520×1080 pixels. From 10 to 13 non-overlapping images of renal cortex per sample are taken using objective ×20 with NA 0.60.

Estimation of interstitial volume fraction is performed using Histometrix-5 software (Kinetic Imaging, GB). Standard grid with vertical density 9 points is applied onto microphotographic image and reference space is defined by marking points falling onto non-relevant structures which should be excluded from estimation: glomerular tuft and space, large blood vessels. After defining the reference space points that fall onto interstitial space (all non-tubular structures) are marked and counted. Overall 1000-1400 points are counted per each sample. Ratio between number of points corresponding to interstitial space and total number of points within reference space provides cortical interstitial volume fraction.

### Example 12

### Total Kidney Volume Estimation

The stereological Cavalieri method of volume estimation was employed for the assessment of renal tissue volume (H. J. G. Gundersen et al. Some new, simple and efficient stereological methods and their use in pathological research and diagnosis. APMIS 96:379-394. 1988; C. V. Howard, M. G. Reed. Unbiased stereology. Three-dimensional measurement in microscopy. BIOS Scientific Publishers, 1998; Mouton P. R. Principles and practices of unbiased stereology. The John Hopkins University Press. 2002). Cavalieri's method allows unbiased and efficient estimation of volume of any structure independently of its shape by measuring areas of parallel sections separated by a known distance. The volume of the structure of interest results from summing up the areas of all sections and multiplying this figure by the distance between sections. Area estimation is done by point counting.

Systematic sections used for the SRFG staining (see Example 10) were used for total renal volume estimation. After elution of bound dyes sections are washed, stained with eosin and photographed at low magnification using stereo microscope Leica MZFLIII equipped with digital camera “Spot-2”. The object micrometer scale is photographed at the same magnification for calibration. Estimation of total volume is performed using Histometrix-5 software (Kinetic Imaging, GB). Standard grid with vertical density 7 points is applied onto microphotographic image and points that fall onto renal tissue are marked and counted. Overall 70-250 points are counted per each sample. Kidney volume is calculated by Histometrix taking into account number of points counted, distance between sections and photographic magnification.

### Example 13

### In Vitro Analysis of the Effect of HNOEL-Iso on Transfected Cells

To evaluate the effect of over-production of HNOEL-iso on fibrosis development in vitro, stable cell lines expressing the gene were established.

2 independent populations of Rat1 fibroblasts, stably transfected with empty vector (PIRES puro) and 2 independent populations of Rat1 fibroblasts, stably transfected with rat-HNOEL pIRES puro were established. For the evaluation of HNOEL-iso effect, TGF-betastimulation was used and the collagen amount and the rate of proliferation of the over-expressors was monitored.

All Rat1 fibroblasts were seeded in 24-well tissue culture plates at plating density of 1×10⁴ in 1 ml of DMEM supplemented with 10% PBS. Puromycin was added at final concentration of 1.9 μg. Cells were grown until subconfluent state (during 96 hours). Then medium was replaced with DMEM, containing 0.1% of BSA. TGF-β of Biotest was added at concentrations of 0.2 ng; 0.5 ng; 1 ng and 2 ng/ml and cells were incubated for additional 72 hours.

At the end of the incubation cells were fixed with the mixture of picric acid, formaldehyde and acetic acid and stained with Sirius Red dye reagent for 1 hour. Then the stained material was dissolved in 0.1N sodium hydroxide (non-bound dye was previously removed with 0.01N hydrochloric acid), the dye solution was transferred to 96-well plates and the optical density (OD) measured at 530 nm against sodium hydroxide as a blank.

To determine cell number in each well, fibroblasts were detached by trypsinization and counted with a haemocytometer.

### Results:

The number of cells in wells with nontransfected cells or cells with empty vector was 3-4 times higher than in cells transfected with HNOEL (the same results were obtained in absolute numbers of O.D.).

It is therefore proposed, without being bound by theory, that overexpression of HNOEL in Rat1 cells leads to the growth arrest of the fibroblasts.

At the same time collagen/10⁴ cells in Rat1 fibroblasts transfected with HNOEL-iso gene provided 1.5-1.7-fold increase in comparison with nontransfected cells or in cells transfected with empty vector. Therefore, the inventors concluded that the HNOEL-iso is a profibrotic gene when over-expressed in fibroblasts treated with TGFbeta.

### Example 14

### Establishment of Polyclonal Antibodies Against HNOEL-Iso

Polyclonal antibodies against two peptides located in the rat HNOEL polypeptide were raised by methods well known in the art:

peptide 1: Ac-CQDQS SRHAA ELRDF KNK-NH₂, located at amino acid residues 44-61
Peptide 2: Ac-LDPQT LDTEQ QWDTP C—NH₂, located at amino acid residues 301-316.

These antibodies recognized a strong-65 kDa protein when the cDNA of HNOEL was overexpressed in cultured cells and recognized a similar sized protein in both rat and mouse kidneys.

### Example 15

### Production and testing of siRNA against HNOEL-iso

We have identified (essentially using known methods as described above) and cloned siRNA sequences for HNOEL. The following 5 siRNA were cloned (all matching gi/276605271 which is the rat HNOEL gene).

1. 5′-GATCCTGAAGCGGTTTGGT-3′
2. 5′-TGAGAAATACGATATGGTG-3′
3. 5′-GATCTACGTGTTAGACGGC-3′
4. 5′-AGAAACTTGGCTAGACACAAA-3′
5. 5′-AGATGGAAAATAGGAGAGTGC-3′

A series of experiments was performed using the above siRNAs:

A. Expression of HNOEL: In cells which express HNOEL endogenously (Rat1 cells), it was shown that the expression of HNOEL was decreased 40-70% when any one of the above siRNAs was transiently transfected into the cells. This was determined on the mRNA level, as tested by semi-quantitative RT-PCR. These experiments were repeated with cells which over-express exogenous HNOEL (kidney epithelial cells strain 293) with essentially the same results.

B. Proliferation rate: Stable clones in Rat1 cells were established which expressed either siRNA #1 and siRNA #2 above (as verified by RT-PCR), resulting in reduction of approximately 50%-60% in HNOEL expression. It was shown that siRNA#1 and also siRNA#2 expressing clones exhibited a reduction in proliferation rate as compared to the empty-vector transfected cells.

C. TGF-beta treatment: Normally, stimulation of Rat1 cells with TGF-beta causes accumulation of fibronectin. The cells described in (B) above, i.e. siRNA #1 and siRNA#2 expressing cells, show reduction in fibronectin accumulation in response to TGF-beta stimulation, as observed both after 24 hr and 48 hr following TGF-beta treatment, compared to the empty-vector cells

These results strongly suggest that inhibition of HNOEL expression may have a beneficial effect on the development of fibrosis.

Similar methods to those described above may be used to produce siRNA to human HNOEL, and this siRNA may be used as a human therapeutic to treat fibrosis.

### Example 16

### Development of Chronic Renal Insufficiency in HNOEL Transgenic Mice

To evaluate the role of HNOEL is spontaneous development of CRI, one line of the transgenic mice overexpressing HNOEL (see Example 3) was studied. This is the same line which was studied in the UUO model (Example 8). Transgenic mice and their wild type littermates, 19 Males, (9 TG and 10 WT mice) were followed for 24 weeks. Clinical signs were taken and body weight was measured (every 4 weeks).

At the end of the study 24 hr urine collection was performed (using metabolic cages) and urine volume, urine creatinine and plasma creatinine levels were measured.

Creatinine clearance was calculated using the following formula—

CrCl=(Ucr)(Uv24hr)/(Pcr)

Upon sacrifice both kidneys were removed and weighed.

Hydroxyproline/mg of dry weight was measured as a marker for fibrosis

Statistical analysis of results was performed using Two Way Nested ANOVA.

A summary of the results is provided in Table 7 below:

TABLE 7
Parameters of WT (N = 10) and HNOEL TG (N = 9) mice

| | WT | TG | | |
|---|---|---|---|---|
| parameters | age-24 weeks | age-24 weeks | p-values | fold change |
| Body weight, g (SD) | 37.43 (2.59) | 37.2 (1.99) | 0.832527775 | |
| Kidney Weight, g (SD) | 0.269 (0.017) | 0.237 (0.02) | 0.001879368 | 12% decrease |
| Kidney wt/Body wt, 10-3 | 7.2 (0.46) | 6.39 (0.56) | 0.002955369 | 12% decrease |
| (SD) | | | | |
| Plasma creatinine mg/dl | 0.395 (0.0639) | 0.4233 (0.0616) | 0.340617562 | |
| (SD) | | | | |
| Urine creatinine mg/dl (SD) | 37.509 (11.213) | 40.08 (6.750) | 0.356365562 | |
| creatinine clearance (SD) | 0.071 (0.023) | 0.043 (0.024) | 0.023337081 | 40% decrease |
| HP content | 1.838 (0.252) | 2.269 (0.202) | 0.000516524 | 20% increase |
| Macro-morphology | Normal | Normal | | |

We concluded that overexpression of HNOEL in the kidney results in a dramatic phenotype whereby not only pathological changes are observed (elevation in hydroxyproline content together with decrease in kidney/body weight) but these are also manifested by reduction in glomerular filtration rate observed by reduced creatinine clearance levels.

1-35. (canceled)

36. A method for the treatment of a fibrosis related pathology in a subject in need of such treatment comprising administering to the subject an amount of an inhibitor of HNOEL-iso polypeptide sufficient to effect a substantial inhibition of the HNOEL-iso polypeptide so as to thereby treat the subject.

37. A method of claim 36 wherein the fibrosis related pathology is liver fibrosis.

38. A method of claim 36 wherein the fibrosis related pathology is pulmonary fibrosis.

39. A method of claim 36 wherein the fibrosis related pathology is cardiac fibrosis.

40. A method of claim 36 wherein the fibrosis related pathology is kidney fibrosis.

41. A method of claim 36 wherein the fibrosis related pathology is scarring.

42. A method of claim 36 wherein the fibrosis related pathology is selected from nephropathy, chronic renal insufficiency, chronic renal failure and glomerulosclerosis.

43. The method of claim 42 wherein the nephropathy is diabetic nephropathy.

44. The method of claim 36 wherein the inhibitor is an antibody which binds specifically to HNOEL-iso polypeptide.

45. The method of claim 36 wherein the inhibitor is an siRNA which is specific to HNOEL-iso polynucleotide.

46. The method of claim 36 wherein the inhibitor is an antisense oligonucleotide corresponding to HNOEL-iso polynucleotide.

47. A pharmaceutical composition for the treatment of fibrosis related pathology comprising as an active ingredient an inhibitor which inhibits production of HNOEL-iso polypeptide together with a pharmaceutically acceptable carrier.

48. The pharmaceutical composition of claim 47 wherein the fibrosis related pathology is selected from liver fibrosis, pulmonary fibrosis and cardiac fibrosis.

49. The pharmaceutical composition of claim 47 wherein the fibrosis related pathology is kidney fibrosis.

50. The pharmaceutical composition of claim 47 wherein the fibrosis related pathology is scarring.

51. The pharmaceutical composition of claim 47 wherein the fibrosis related pathology is selected from nephropathy, in particular diabetic nephropathy, chronic renal insufficiency, chronic renal failure and glomerulosclerosis.

52. The pharmaceutical composition of claim 47 wherein the inhibitor is an antibody which binds specifically to HNOEL-iso polypeptide.

53. The pharmaceutical composition of claim 47 wherein the inhibitor is an siRNA which is specific to HNOEL-iso polynucleotide.

54. The pharmaceutical composition of claim 47 wherein the inhibitor is an antisense oligonucleotide corresponding to HNOEL-iso polynucleotide.

55. A method for the treatment of disease selected from osteoarthritis, osteoporosis, other bone disease and cardiovascular disease in a subject in need of such treatment comprising administering to the subject an amount of an inhibitor of HNOEL-iso polypeptide sufficient to effect a substantial inhibition of the HNOEL-iso polypeptide so as to thereby treat the subject.
